(19) United States
(12) Patent Application Publication
(10) Pub. No.: US 2026/0363685 A1
(43) Pub. Date: Nov. 19, 2026

(54) BENDABLE ARTICULATED STRUCTURES AND ANNULAR MEMBERS THEREFOR

(71) Applicant: MOMENTIS SURGICAL LTD., Or-Yehuda (IL)

(72) Inventor: Yiftah NETA, Or Yehuda (IL)

(21) Appl. No.: 19/167,372

(22) PCT Filed: Mar. 25, 2024
(86) PCT No.: PCT/IL2024/050301

§ 371 Date: Sep. 21, 2025

Related U.S. Application Data
(60) Provisional application No. 63/454,695, filed on Mar. 26, 2023.

(51) Int. Cl. A61B 17/00

(52) U.S. Cl. CPC A61B 17/00234; A61B 2017/00314; A61B 2017/00327

(57) ABSTRACT

An articulated arm comprises a tubular structure that includes a bendable section comprising a concatenated annular members. Each annular member comprises a first pair of axial projections and a corresponding first pair of recesses axially aligned therewith. Each projection is shaped to have an apex point at a vertex of a convex angle, and each corresponding recess is shaped to surround a volume having an apex point at a vertex of a concave angle within the recess. The annular members are arranged in the bendable section such that for each pair of consecutive annular members, the projections of a first annular member are pivotably engaged with corresponding recesses of a second annular member. Bending the bendable section includes pivoting respective concave angles of the recesses of the second annular member about the apex points of the projections of the first annular member.

### FIELD AND BACKGROUND OF THE INVENTION

The present invention relates to articulated arms comprising tubular structures that include bendable sections comprising concatenated annular members.

Articulated arms comprising tubular structures having longitudinal, i.e., axially-oriented, channels for passage therethrough of cables and wires have been introduced. In some cases the tubular structures include concatenated annular members formed with corresponding projections and recesses to facilitate pivoting of the annular members and bending of bendable portions of the tubular structures. However, in such cases, the projections and recesses have used naïve, symmetrical designs that cause the interaction between them to suffer from backlash and/or slippage, and to be hindered by excessive friction between neighboring members, all of these potentially affecting the pivoting precision of the articulated arms. There is a need for annular members that reduce or eliminate or reduce the impacts of the drawbacks of the known designs.

### SUMMARY OF THE INVENTION

According to embodiments of the present invention, an articulated arm comprises a tubular structure that includes a bendable section comprising a plurality of concatenated annular members. Each annular member comprises a first pair of axial projections and a corresponding first pair of recesses axially aligned therewith; each projection is shaped to include an apex point defining a vertex of a convex-angle portion of the projection, and each corresponding recess is shaped to surround a volume having an apex point defining a vertex of a concave-angle portion of the recess. The plurality of annular members is arranged in the bendable section such that for each pair of consecutive annular members, the projections of a first annular member are pivotably engaged with corresponding recesses of a second annular member. A pivoting of the first and second annular members relative to each other so as to bend the bendable section includes a pivoting of the respective concave-angle portions of the corresponding recesses of the second annular member about the respective apex points of the projections of the first annular member.

In some embodiments, the apex points of the projections of the first annular member can be in direct contact with the respective vertices of the concave-angle portions of the corresponding recesses of the second annular member when the articulated arm is in an assembled and operative state.

In some embodiments, the apex points of the projections can be in direct contact with the respective vertices of the concave-angle portions when the respective concave-angle portions pivot about the respective apex points of the projections.

In some embodiments, the convex angles of the convex-angle portions of the projections can be smaller than respective conjugate angles of the concave angles of the concave-angle portions of the corresponding recesses.

In some embodiments, the axial projections of the first pair of axial projections can be diametrically opposed from each other.

In some embodiments, the projections can be respectively devoid of axes of symmetry. In some embodiments, the recesses can be respectively devoid of axes of symmetry.

In some embodiments, the pivoting of the first and second annular members relative to each other can be substantially without backlash. In some embodiments, the pivoting of the first and second annular members relative to each other can be substantially without friction in the recesses.

In some embodiments, each annular member can comprise a second pair of axial projections and a corresponding second pair of recesses axially aligned therewith, wherein, for each pair of consecutive annular members, the second-pair projections of the first annular member are slidably engaged with corresponding second-pair recesses of the second annular member to form respective guiding arrangements, a first guiding arrangement being effective to define a pivot limit of the first and second annular members in a direction of the flexing. In some such embodiments, it can be that the pivoting of the first and second annular members relative to each other is not limited by a pivot limit of the pivoting of the concave-angle portions of the corresponding recesses of the second annular member about the respective apex points of the projections of the first annular member. In some embodiments, the axial projections of the second pair of axial projections can be diametrically opposed from each other. In some embodiments, the axial projections of the first and second pairs of axial projections can be distributed evenly around the respective circumferences of each annular member.

In some embodiments, the articulated arm can comprise a first bendable section comprising a respective plurality of concatenated annular members having a first diameter, and a second bendable section comprising a respective plurality of concatenated annular members having a second diameter that is different than the first diameter.

In some embodiments, the pivoting of the first and second annular members relative to each other can include a maximum relative pivoting of at least 5°, or at least 6°, or at least 7°. In some such embodiments, it can be that the maximum relative pivoting is no more than 12°, or no more than 10°, or no more than 8°.

In some embodiments, bending either one of the first or second bendable sections can include bending the respective bendable section by at least 150°, or at least 180°. In some embodiments, bending at least one of the first and second bendable sections can include bending the respective bendable section by at least 210°.

According to embodiments of the present invention, an articulated arm comprises a tubular structure that includes a bendable section comprising a plurality of concatenated annular members. Each annular member comprises one or more axial pivot-projections and one or more corresponding pivot-recesses axially aligned therewith, each of the one or more pivot-projections being shaped to include an apex point defining a vertex of a convex-angle portion of the pivot-projection, each one of the one or more corresponding pivot-recesses being shaped to surround a volume having an apex point defining a vertex of a concave-angle portion of the pivot-recess. The plurality of annular members is arranged in the bendable section such that for each pair of consecutive annular members, the one or more pivot-projections of a first annular member are pivotably engaged with the one or more corresponding pivot-recesses of a second annular member. A pivoting of the first and second annular members relative to each other so as to bend the bendable section includes a pivoting of the respective concave-angle portions of the one or more corresponding pivot-recesses of the second annular member about the respective apex points of the one or more pivot-projections of the first annular member.

In some embodiments, the one or more axial pivot-projections can include exactly one pivot-projection, and/or the one or more corresponding recesses can include exactly one corresponding pivot-recess, and/or the exactly one pivot-projection of the first annular member can be pivotably engaged with the exactly one corresponding pivot-recess of the second annular member.

In some embodiments, the one or more axial pivot-projections can include exactly one pivot-projection, and/or the one or more corresponding pivot-recesses can include exactly two pivot-recesses diametrically opposed from each other, and/or the exactly one pivot-projection of the first annular member can be pivotably engaged with one of the two pivot-recesses of the second annular member.

In some embodiments, for each pivot-recess of the second annular member that is pivotably engaged with a respective apex point of a pivot-projection of the first annular member, the respective apex is in direct contact with the respective vertex of the concave-angle portion of the pivot-recess when the articulated arm is in an assembled and operative state.

In some embodiments, the apex points of the pivot-projections can be in direct contact with the respective vertices of the concave-angle portions when the respective concave-angle portions pivot about the respective apex points of the pivot-projections.

In some embodiments, the convex angles of the convex-angle portions of the pivot-projections can be smaller than respective conjugate angles of the concave angles of the concave-angle portions of the corresponding pivot-recesses.

In some embodiments, the one or more axial pivot-projections can include exactly two pivot-projections that are diametrically opposed from each other, and/or the one or more corresponding pivot-recesses can include exactly two pivot-recesses that are diametrically opposed from each other.

In some embodiments, the pivot-projections can be respectively devoid of axes of symmetry. In some embodiments, the pivot-recesses can be respectively devoid of axes of symmetry.

In some embodiments, the pivoting of the first and second annular members relative to each other can be substantially without backlash. In some embodiments, the pivoting of the first and second annular members relative to each other can be substantially without friction in the pivot-recesses.

In some embodiments, each annular member can comprise one or more axial guide-projections and one or more corresponding guide-recesses axially aligned therewith, wherein the one or more guide-projections of the first annular member are slidably engaged with corresponding one or more guide-recesses of the second annular member to form one or more respective guiding arrangements, a first guiding arrangement being effective to define a pivot limit of the first and second annular members in a direction of the flexing.

In some embodiments, it can be that the pivoting of the first and second annular members relative to each other is not limited by a pivot limit of the pivoting of the concave-angle portions of the corresponding one or more pivot-recesses of the second annular member about the respective apex points of the one or more pivot-projections of the first annular member.

In some embodiments, the one or more axial guide-projections can include exactly two pivot-projections that are diametrically opposed from each other, and the one or more corresponding guide-recesses include exactly two pivot-recesses that are diametrically opposed from each other.

In some embodiments, the articulated arm can comprise a first bendable section comprising a respective plurality of concatenated annular members having a first diameter, and a second bendable section comprising a respective plurality of concatenated annular members having a second diameter that is different than the first diameter.

In some embodiments, the pivoting of the first and second annular members relative to each other can include a maximum relative pivoting of at least 5°, or at least 6°, or at least 7°. In some such embodiments, the maximum relative pivoting can be no more than 12°, or no more than 10°, or no more than 8°.

In some embodiments, bending either one of the first and second bendable sections can include bending the respective bendable section by at least 150°, or at least 180°. In some such embodiments, bending at least one of the first and second bendable sections can include bending the respective bendable section by at least 210°.

According to embodiments of the present invention, an annular constituent element for a bendable section of a tubular structure comprises one or more axial projections and one or more corresponding recesses axially aligned therewith. Each of the one or more projections is shaped to include an apex point defining a vertex of a convex-angle portion of the projection. Each one of the one or more corresponding recesses is shaped to surround a volume having an apex point defining a vertex of a concave-angle portion of the recess.

In some embodiments, it can be that the one or more axial projections comprise respective corresponding inner and outer surfaces, and/or that each respective outer surface has an area smaller than an area of a corresponding axially-aligned-therewith recess.

In some embodiments, the convex angles of the respective convex-angle portions of the one or more projections can be smaller than respective conjugate angles of the concave angles of the respective concave-angle portions of the one or more corresponding recesses.

In some embodiments, the one or more axial projections can include exactly two projections that are diametrically opposed from each other, and/or the one or more corresponding recesses can include exactly two recesses that are diametrically opposed from each other.

In some embodiments, the one or more axial projections can be respectively devoid of axes of symmetry. In some embodiments, the one or more corresponding recesses can be respectively devoid of axes of symmetry.

A method is disclosed, according to embodiments of the present invention, for producing a bendable section for a tubular structure. The method comprises at least one of cutting, carving and sculpting a lengthwise portion of the tubular structure to form a plurality of concatenated annular members. Each annular member is shaped by the at least one of cutting, carving and sculpting to comprise one or more axial pivot-projections and one or more corresponding pivot-recesses axially aligned therewith, each of the one or more pivot-projections being shaped to include an apex point defining a vertex of a convex-angle portion of the pivot-projection, each one of the one or more corresponding pivot-recesses being shaped to surround a volume having an apex point defining a vertex of a concave-angle portion of the pivot-recess. For each pair of consecutive shaped annular members, the one or more pivot-projections of a first annular member are pivotably engaged with the one or more corresponding pivot-recesses of a second annular member.

In some embodiments, a pivoting of the first and second annular members relative to each other so as to bend the bendable section can include a pivoting of the respective concave-angle portions of the one or more corresponding pivot-recesses of the second annular member about the respective apex points of the one or more pivot-projections of the first annular member.

In some embodiments, it can be that the one or more axial pivot-projections include exactly one pivot-projection, and/or that the one or more corresponding recesses include exactly one corresponding pivot-recess, and/or that the exactly one pivot-projection of the first annular member is pivotably engaged with the exactly one corresponding pivot-recess of the second annular member.

In some embodiments, it can be that the one or more axial pivot-projections include exactly two pivot-projections that are diametrically opposed from each other, and/or that the one or more corresponding recesses include exactly two corresponding pivot-recesses that are diametrically opposed from each other, and/or that the exactly two pivot-projections of the first annular member are respectively pivotably engaged with the exactly two corresponding pivot-recesses of the second annular member.

In some embodiments, the convex angles of the convex-angle portions of the pivot-projections can be smaller than respective conjugate angles of the concave angles of the concave-angle portions of the corresponding pivot-recesses.

In some embodiments, the at least one of cutting, carving and sculpting can include laser-cutting.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is a schematic plan view of a portion of an articulated arm comprising a tubular structure, according to embodiments of the present invention;

FIG. 2A shows a bendable portion of a tubular structure of an articulated arm, according to embodiments of the present invention;

FIG. 2B shows details of a plurality of annular members of the bendable portion of FIG. 2A, according to embodiments of the present invention;

FIG. 2C shows a bendable portion of a tubular structure in a bent state, according to embodiments of the present invention.

FIG. 2D is a schematic perspective view of annular members of a bendable portion, according to embodiments of the present invention;

FIGS. 3A and 3B are schematic illustrations of an annular member, respectively showing the side having axial projections and the side having axial recesses, according to embodiments of the present invention;

FIGS. 4, 5 and 6 are schematic illustrations showing features of axial projections and corresponding recesses of annular members, according to embodiments of the present invention;

FIGS. 7A, 7B and 7C are schematic illustrations showing features and components of annular members at successive stages of pivoting, according to embodiments of the present invention;

FIG. 8 is a schematic perspective view of annular members of a bendable portion, according to embodiments of the present invention;

FIGS. 9A and 9B are schematic illustrations of axial projections and corresponding recesses of annular members of a bendable portion, wherein each axial projection comprises a projection-recess surrounding a volume having an apex, and each axial recess comprises a recess-projection having an apex, according to embodiments of the present invention;

FIGS. 10A, 10B and 11 are schematic illustrations showing features of axial projections and corresponding recesses of annular members of a bendable portion, according to embodiments of the present invention;

FIG. 12 is a schematic perspective view of a plurality of annular members of a bendable portion, according to embodiments of the present invention;

FIG. 13 illustrates a pivot limit of annular members of a bendable portion, according to embodiments of the present invention;

FIG. 14 shows a flowchart of a method for producing a bendable section for a tubular structure; and

FIG. 15 is a schematic perspective view of a plurality of annular members of a bendable portion produced by the method of claim 14, according to embodiments of the present invention.

It should be understood that the descriptions of specific embodiments of the invention are provided for illustrative purposes only and do not serve as a limitation on the scope of the invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

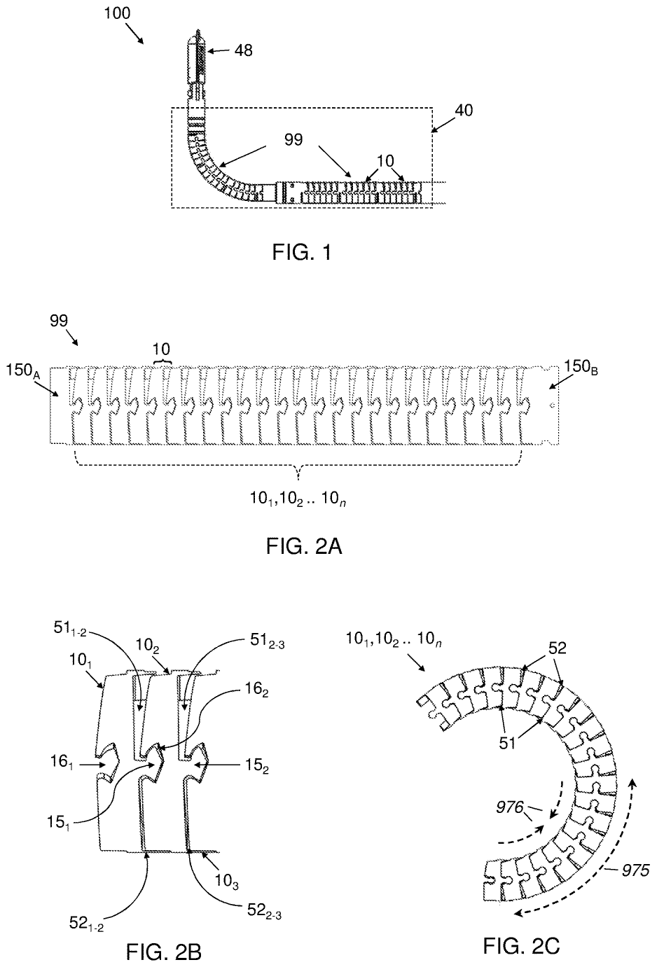

Referring to the figures and in particular to FIG. 1, an articulated arm 100 is provided according to embodiments. The arm 100 includes a tubular structure 40 comprising, in a non-limiting example, two bendable portions 99 of differing diameters. Each bendable portion 99 comprises a concatenated plurality of annular members 10. In some embodiments, the different-diameter annular members 10 of the two bendable portions 99 have the same thickness as each other, i.e., the same dimension in the axial direction. In some embodiments, the different-diameter annular members 10 of the two bendable portions 99 are proportionally dimensioned, i.e., the smaller-diameter member 10 of the different-diameter annular members 10 is a proportionately ‘shrunken’ version of the larger-diameter member 10 of the two.

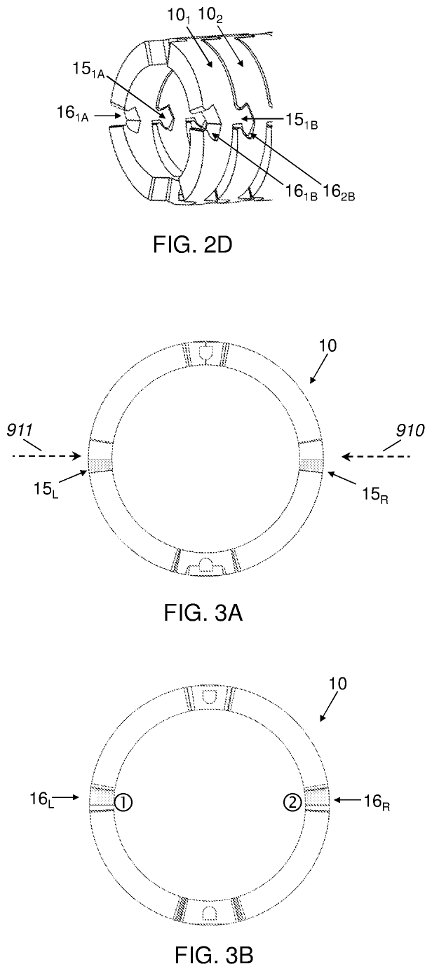

FIG. 2A shows an example of a bendable portion 99 according to embodiments. The bendable portion 99 comprises n annular members 10 and two end elements 150_{A}, 150_{B} and arranged. The number n can be any integer according to the length requirements of the bendable portion 99, of the tubular section 40, or of the articulated arm 100. In some embodiments, the number n is based on or related to the maximum bend required for a bendable portion 99 divided by the maximum pivot angle of one annular member 10 relative to another. For example, if a bendable portion 99 will be required to double back on itself, i.e., bend through at least 180°, and the maximum pivot angle of adjacent or consecutive annular members 10 is 7.8°, then n can be made equal to 23, 24, 25 or more. In embodiments, the maximum pivot angle of adjacent or consecutive annular members 10 is at least 5°, or at least 6°, or at least 7°, and the maximum pivoting angle is not more than 12°, or not more than 10°, or not more than 8°. In embodiments, any bendable section can be flexed by at least 150°, or at least 180°. In an arm 100 having two different-diameter bending portions 99 as shown in the example of FIG. 1, the two bending portions 99 can have different minimum aggregate bending angles, e.g., where a first bending portion 99 can be flexed by at least 180° and a second bending portion 99 can be flexed by at least 210°.

In some embodiments, the different-diameter annular members 10 of the two bendable portions 99 of FIG. 1 are configured to flex the respective bendable portions 99 to have a central longitudinal axis with a radius of not more than 20 mm, or not more than 15 mm, or not more than 13 mm. In embodiments, the respective axes of the two bending portions 99 have the same minimum radius when flexed to a respective maximum extent as in, for example, FIG. 2C.

FIG. 2B shows a side view of multiple annular members 10 of a bendable portion 99 in greater detail. The annular members 10 are concatenated and serve as ‘links’ making up the bendable portion 99, or at least making up an annular part of the bendable portion 99. Each annular member 10 includes an axially-extending projection 15 and a corresponding axial recess 16. The projections 15 and recesses 16 are designed so that projections 15 fit within, and can pivot within, corresponding recesses 10 of consecutive annular members 10. In the example of FIG. 2B, projection 151 of the first annular member 10₁ fits within the volume of the recess 162 of the second annular member 10₂ so as link the consecutive annular members 10₁, 10₂ to each other. The first annular member 10₁ is thus linked with the second annular member 10₂.

The annular members 10 are shaped to enable pivoting of annular members 10 relative to each other. As can be seen in FIG. 2B, the annular members 10 are narrower on one side (the ‘upper side’ in the view shown in FIG. 2B), i.e., around a first portion of the circumference, than on the other side (the ‘lower side’ in FIG. 2B). The shape of the annular members 10 leaves a large gap 51 when the bendable portion is not bent, e.g., gap 51₁₋₂between annular member 10₁ and annular member 10₂, or gap 51₂₋₃ between annular member 10₂ and annular member 10₃ on the ‘upper side’. On the ‘lower side’, gap 52, e.g., gap 52₁₋₂ between annular member 10₁ and annular member 10₂, or gap 52₂₋₃ between annular member 10₂ and annular member 10₃, is substantially smaller in the unbent state of the bendable portion 99.

FIG. 2C shows a bendable portion 99 in a fully bent (flexed) state, such that every pair of consecutive annular members 10ᵢ, 10ᵢ₊₁ out of the plurality of annular members 10₁ . . . 10ₙ can be pivoted relative to each other in the direction(s) shown by arrows 975 and 976 in FIG. 2C, to at least partly close the gaps 51, and open up the gaps 52. When the annular members 10ᵢ, 10ᵢ₊₁ pivot, the projection of annular member 10ᵢ pivots within the corresponding recess of the annular members 10_{i+I}. In the example of FIG. 2C, all of the annular members 10 of the bendable portion 99 are shown as being pivoted so as to achieve a total bend of more than 210°.

FIG. 2D shows a side view of multiple annular members 10 of a bendable portion 99 in perspective view, in which it can be seen that each annular member 10 is shaped to include a pair of axially extending projections 15, and a corresponding pair of axially oriented recesses 16. Specifically, the first annular member 10₁ in FIG. 2D includes projections 15_{1A} and 15_{1B}, and the second annular member 10₂ includes projections 15_{2A} and 15_{2B}. According to some embodiments, the projections 15_{iA} and 15_{iA} of an annular member 10ᵢ are diametrically opposed from each other, and the corresponding recesses 16_{iA} and 156_{iA} are diametrically opposed from each other. When consecutive annular members 10ᵢ, 10ᵢ₊₁ are concatenated/linked as shown in FIG. 2D, respective projections 15_{iA} are axially aligned with and pivotably engaged with corresponding recesses 16_{iA}, while respective projections 15_{iB} are axially aligned with and pivotably engaged with corresponding recesses 16_{iB}.

In embodiments, individual concatenated annular members 10 cannot be non-destructively separated from each other. As can be seen, inter alia, in FIGS. 2B and 2D, the respective shapes of the projections 15 and recesses 16, e.g., in combination with selection of a construction material of sufficient rigidity, are selected so as to be effective to prevent non-destructive separation of concatenated annular members 10 with a longitudinal force or twisting force. The process of selecting a suitable design of axial projections 15 and recesses 16, along with selecting a suitable construction material for the annular members 10, can consist of a separation analysis and/or physical test to check whether the design of the axial projections 15 and recesses 16 is sufficiently robust, and whether the material is not overly pliable, so as not to allow forced but non-destructive separation of concatenated annular members 10. Further, the protrusions axial projections 15 and recesses 16 are shaped so as not to allow separation of concatenated annular members 10 by lateral sliding. FIGS. 3A and 3B show elevation views of an annular member 10, with FIG. 3A showing the side having axial projections 15 and FIG. 3B showing the side having axial recesses 16. As seen in both of these figures, the axial projections 15 and recesses 16 have substantially the same radial thickness of the annular member 10, which means that each of the projections 15 and recesses 16 is wider, i.e., circumferentially longer, on the outside of the annular member 10 than on the inside. In the case of a left-to-right lateral force, e.g., as indicated by arrow 911, the left-side projection 15_{L} of FIG. 3A would not be able to slide laterally out of the left-side recess 16_{L} of FIG. 3B, and separation of the annular members 10 would be prevented by the narrowing of the left-side recess 16_{L} at point \textcircled{1}. In the case of a right-to-left lateral force, e.g., as indicated by arrow 910, the right-side projection 15_{R} of FIG. 3A would not be able to slide laterally out of the right-side recess 16_{R} of FIG. 3B, and separation of the annular members 10 would be prevented by the narrowing of the right-side recess 16_{R} at point \textcircled{2}.

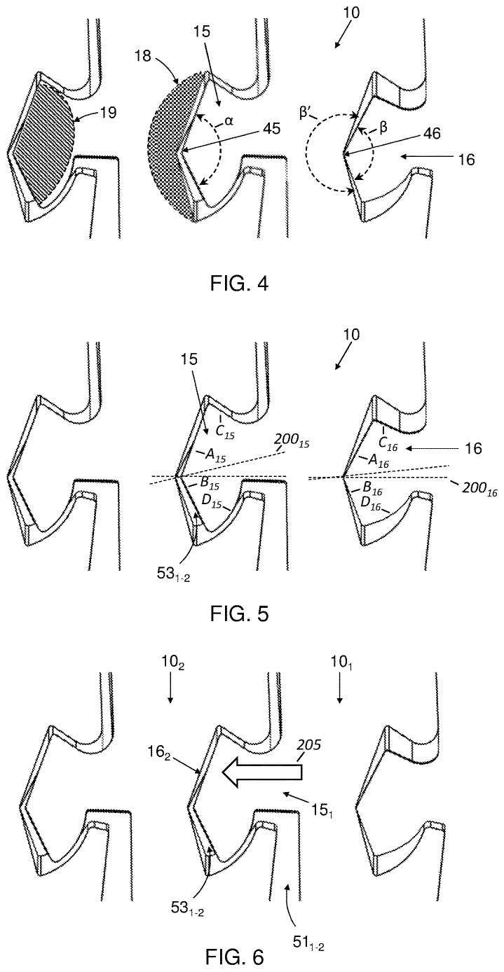

We now refer to FIGS. 4, 5 and 6, which disclose certain structural and functional attributes of the axial projections 15 and corresponding recesses 16 of annular members 10 according to embodiments.

As can be seen in FIG. 4, the projection 15 of the annular member 10 is shaped to include an apex point 45. The apex point 45 defines a vertex of a convex-angle portion 19 of the projection 15 defined at the apex point 45 by convex angle α. The convex-angle portion 19 shown in FIG. 4 is an illustrative example, and in other examples, the convex-angle portion 19 can have a different shape, i.e., include a differently shaped portion of the projection 15 including the apex point 45 and the rays extending therefrom (e.g., A₁₅ and B₁₅ in FIG. 5).

The corresponding recess 16 is shaped to surround a volume having an apex point 46. The apex point 46 defines both a vertex of a convex-angle volume defined at the apex point 46 by convex angle β, and a vertex of a concave-angle portion 18 partly circumscribing the recess 16 that subsumes the convex-angle volume and that is defined at the apex point 46 by concave angle β′. The concave-angle portion 18 ‘of the recess 16’, meaning ‘partly circumscribing the recess 16’, that is shown in FIG. 4 is an illustrative example, and in other examples, the concave-angle portion 18 can have a different shape, i.e., include a differently shaped portion of the annular member 10 partly circumscribing the recess 16 and including the apex point 46 and the rays extending therefrom (e.g., A₁₆and B₁₆ in FIG. 5).

In embodiments, the convex angle α of the convex-angle portion of the projection 15 is greater than the convex angle β, which is the conjugate angle of the concave angle β′ of the concave-angle portion of the corresponding recess 16. In embodiments, all of the annular members 10 of a given bendable portion 99 have substantially identical designs. Referring again to FIG. 2A, end members 150 of a bendable portion 99 may include projections 15 and/or recesses 16 to link to, i.e., be pivotably engaged with, the first or last annular member 10 in a concatenated plurality annular members 10.

FIG. 5 schematically illustrates the lack of symmetry in projections 15 and recesses 16 according to embodiments. Axes 200₁₅ that could serve as axes of symmetry are shown as attempting to bisect the projection 15. One axis 200₁₅ passes through the apex point 45 and is parallel to a longitudinal axis (not shown) of the bendable portion 99 and of the linked annular members 10. A second axis 200₁₅ passes through the apex point 45 and the center of the narrow (‘stem’) portion of the projection 15. In either case, the line segments and chords A₁₅, B₁₅, C₁₅, and D₁₅ making up the profile of the convex-angle portion of the projection 15 on both sides of the ‘bisecting’ axes do not exhibit any symmetry—A₁₅, for example, is longer than B₁₅, and D₁₅ is longer and more curved than C₁₅. Axes 200₁₆ that could serve as axes of symmetry are shown as attempting to bisect the recess 15. One axis 200₁₆ passes through the apex point 46 and is parallel to a longitudinal axis (not shown) of the bendable portion 99 and of the linked annular members 10. A second axis 200₁₆ passes through the apex point 46 and the center of the narrow portion of the recess 16. In either case, the line segments and chords A₁₆, B₁₆, C₁₆, and D₁₆ making up the profile of the concave-angle portion circumscribing the recess 16 on both sides of the ‘bisecting’ axes do not exhibit any symmetry—A₁₆, for example, is longer than B₁₆, and D₁₆ is longer and more curved than C₁₆.

In embodiments, the pivoting of the one of more recesses 16 of a given annular member 10 about corresponding projections 15 of a neighboring annular member 10 optimally takes place when the apex points 45 of the convex-angle portions 19 are in direct contact with the corresponding concave-angle portions 18 at their respective apex points 46. In other words, in such embodiments, slack is removed between the annular members 10 in order to produce direct contact between consecutive annular members 10. Inter alia, such direct contact ensures that the apex points 45, 46 combine to form a pivot point. In some embodiments, it can be desirable for line segment A₁₅ of each projection 15 to be substantially flush against the line segment A₁₆ of each corresponding recess 16. The concave-angle portion 18 of the recess 16 thus pivots precisely about the apex point 45 of the convex-angle portion 19 of the projection 15. In order to create and maintain such direct contact, an axial force can be applied, e.g., a force indicated by arrow 205 in FIG. 6. In embodiments, when there is direct contact direct contact between consecutive annular members 10 as described, pivoting of the first and second annular members 10₁, 10₂ relative to each other is substantially without backlash or slippage that might be induced in design where pivoting occurs with slack between projection 15 and recess 16, i.e., at the apex points 45, 46. It can also be understood that because the extent of direct contact between projection 15 and recess 16 during pivoting is limited to the contact between the apex points 45, 46, the pivoting is substantially without friction. Elimination of backlash and/or friction can contribute to a more precise and controllable flexing of the articulate arm 100.

As is shown schematically in FIG. 6, the force 205 is effective to drive the apex point 45 of the convex-angle portion 19 of the projection 15 to come into contact with the apex point 46 of the corresponding concave-angle portions 18 of the recess 16. In a non-limiting example, a cable (not shown) passes through each of one or more axially-oriented and axially-aligned holes 43 in the annular members 10 of a bendable portion 99.

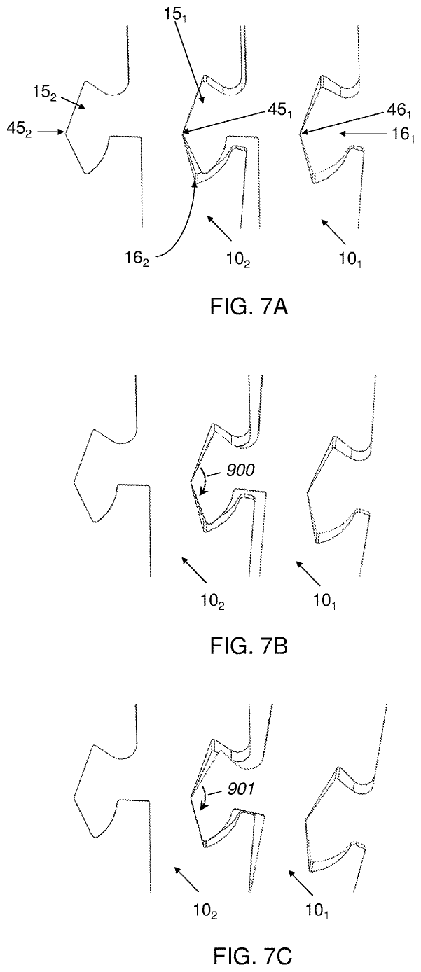

FIG. 7A shows the first and second annular members 10₁, 10₂ of FIG. 6, with the apex point 45₁ of the convex-angle portion 19 of the projection 15₁ of the first annular member 10₁ being in contact with the apex point 46₁ of the concave-angle portion 18₂ of the recess 16₂ of the second annular member 10₂ (reference numbers 46₁ and 18₁ are not shown in FIG. 7A due to lack of space).

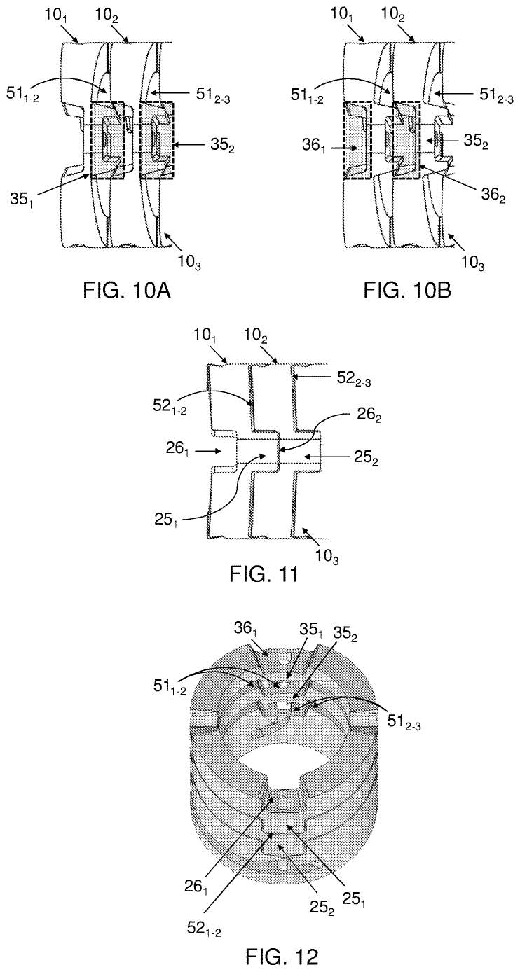

FIGS. 7B and 7C illustrate the pivoting of the respective concave-angle portion 18₂of the corresponding recess 16₂ of the second annular member 10₂ about the respective apex point 45₁ of the projection 15 of the first annular member 10₁. It should be noted that for the purposes of this disclosure, expressing that recesses 16 and respective 46 of respective concave-angle portion 18 pivot about apexes 45 of projections 15 is equivalent to expressing that apexes 45 of projections 15 pivot within recesses 16, as all ‘pivoting’ is relative, and neither the projections 15 nor the recesses 16 are fixed in space. In the non-limiting example, of FIG. 7B, as indicated by arrow 900, the pivoting comprises about 5° of pivot. In the non-limiting example, of FIG. 7C, as indicated by arrow 901, the pivoting comprises about 11° of pivot. In other examples, the maximum pivot range can be larger or smaller than 11° of pivot. In other examples, the maximum pivot range is determined by other design elements, such as the design of guiding projections and recesses as discussed hereinbelow with respect to FIGS. 10A, 10B, 11, 12 and 13. In such other examples, the pivoting range shown in FIG. 7C can be constrained and the projection line segment B₁₅ (see FIG. 5) does not contact the recess line segment B₁₆ to fully close the gap 53 between Bᵢₛ and B₁₆.

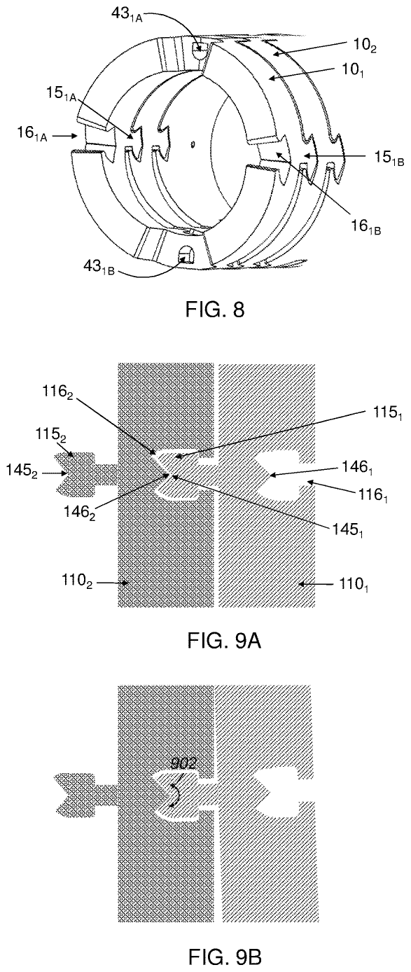

FIG. 8 shows an exemplary pair of holes 43_{1A}, 43_{1B} in the annular member 10₁. According to the example, the cable is operable as an ‘actuator cable’ to transmit a mechanical force through the bendable portion 99 to an end effector 48 (shown in FIG. 1). Making the cable taut before operating the articulated arm 100 can serve to remove the slack between consecutive annular members 10 in a bendable portion 99 so as to put the apex points 45 of the convex-angle portions 19 in direct contact with the corresponding concave-angle portions 18 of the recesses 16 at their respective apex points 46. In another example, the one or more cables are operable to apply different forces to different portions of the annular members 10 so as to cause pivoting of annular members 10 relative to each other and bending of the bendable portion 99. In another example, the cable transmits electrical power. In yet another example, s cable functions primarily to remove the inter-member slack in the bendable portion 99 and the articulated arm 100. In an additional example, multiple cables can be passed through the holes 43 such that a first cable performs a first function, e.g., eliminating slack in the bendable portion 99 and/or bending the bendable portion 99, and a second cable performs a second function, e.g., operating an end effector. In some embodiments, the articulated arm 100 is configured such that when the respective concave-angle portions 18 pivot about the respective apex points 45 of the projections 15, the apex points 45 of the projections 18 are in direct contact with the respective vertices 46 of the concave-angle portions 18.

FIGS. 9A and 9B are schematic illustrations of concatenated annular members 110₁, 110₂ comprising respective axial projections 115 and corresponding recesses 116 according to some embodiments. The axial projections 115 and corresponding recesses 116 of FIGS. 9A and 9B differ from axial projections 15 and corresponding recesses 16 of, e.g., FIGS. 2B and 2D, in that each axial projection 115 comprises a projection-recess surrounding a volume having an apex 145, and each axial recess 116 comprises a recess-projection having an apex 146. Nonetheless, as indicated in by arrow 902 in FIG. 9B, the two concatenated annular members 110₁, 110₂ are configured to pivot relative to each other as the projection-recess of the axial projection 115₂ of the first annular member 110₁ pivots about the recess-projection of the corresponding recess 116₂ of the second annular member 110₂. In such embodiments, the pivoting can be when the apex 145₁ of projection 115₁ of the first annular member 110₁ contacts the apex 146₂ of the empty volume of the recess-projection of the recess 116₂ of the second annular member 110₂. Any features described with respect to concatenated annular members 10 comprising respective axial projections 15 and corresponding recesses 16 apply, mutatis mutandis, to concatenated annular members 110 comprising respective axial projections 115 and corresponding recesses 116.

We now refer to FIGS. 10A, 10B, 11, 12 and 13.

In embodiments, annular members 10 can be formed to include guiding projections for controlling the pivoting of annular members 10 relative to each other when a bendable portion 99 is flexed. In some embodiments, each member 10 includes a pair of guiding projections that are diametrically opposed to each other, i.e., on radially-opposite sides of the annular member. It can be desirable for the opposing pair of guiding projections to differentially control bending of the bendable portion 99 such that the bendable portion bends more in a first direction than in a second direction. The differential control can be at least partly facilitated by providing pair of guiding projections including a first guiding projection that facilitates more pivoting in its direction, and a different, second guiding projection that facilitates less pivoting in its direction, or that even inhibits pivoting in its direction.

FIGS. 10A and 10B both show the same side view of a plurality of linked annular members 10 similar to FIG. 2B, with the members 10 in the figure rotated 90° to show a first type of axially-oriented guiding projections 35 and corresponding guiding recesses 36. FIG. 10A highlights the first guiding projections 35₁, 35₂ of respective annular members 10₁, 10₂ and FIG. 10B highlights the corresponding first guiding recesses 36₁, 36₂. The guiding projections 35 are shown as generally trapezoidal (with rounded corners) but this is a specific design choice; in some examples the shape can be more rectangular or hemispherical or any other shape that serves the function and/or manufacturing process. Similarly, the large ‘valley’ in the middle of each guiding projection 35 is optional and can be present for any one of a number of design and/or optimization reasons. Inter alia, the valley can be useful for making two contact points out of one guiding projection 35 when the members 10 are pivoted to contact the guiding recess 36 of the adjacent member 10 e.g., in order to make the contact more stable and/or more balanced; and/or for saving mass and material; and/or for optimizing the axial disposition of a hole 43 through which a cable is passed. The guiding recesses 35 are also shown in FIG. 10B as generally trapezoidal as well. It is generally desirable for the guiding projections 35 and the guiding recesses 36 to have complementary shapes that contribute to creating a first guiding arrangement, and, in particular, a stable guiding arrangement. FIG. 11 shows a second side view of a plurality of linked annular members 10, the members 10 in the figure rotated 180° from FIGS. 10A and 10B to show a second type of axially-oriented guiding projections 25 and corresponding guiding recesses 26, which have complementary shapes that contribute to creating a second guiding arrangement.

FIG. 12 shows a top perspective view of a plurality of linked members 10 to show both types of guiding projections 35, 25 and both types of the guiding recesses 36, 26, along with both respective types of gaps 51, 52. The skilled artisan will understand that the larger projection-recess gaps 51 in the first guiding arrangements (relative to the much smaller gaps 52 of the second guiding arrangements) facilitate the bending of the bendable portion 99 and the pivoting of the annular members 10 relative to each other particularly in the direction of the larger gaps 51. In other words, in the exemplary design of FIG. 12, flexing is favored, from a structural perspective, in the direction of the first guiding arrangements which comprise the first types of guiding projections 35 and the first type of guiding recesses 36, in contrast to the second guiding arrangements. The design tends to limit flexing in the direction of the second guiding arrangements, which comprise the second types of guiding projections 25 and the second type of guiding recesses 26, to de minimis pivoting, and even tends to inhibit flexing in that direction. Referring again to FIG. 2C, which shows a bending portion 99 at full flex, the bending portion is flexed in the direction of the larger gaps 51, which are substantially closed by the pivoting evidenced in FIG. 2C, while the small gaps 52 are ‘opened up’ by the flexing in the direction of the first guiding arrangements.

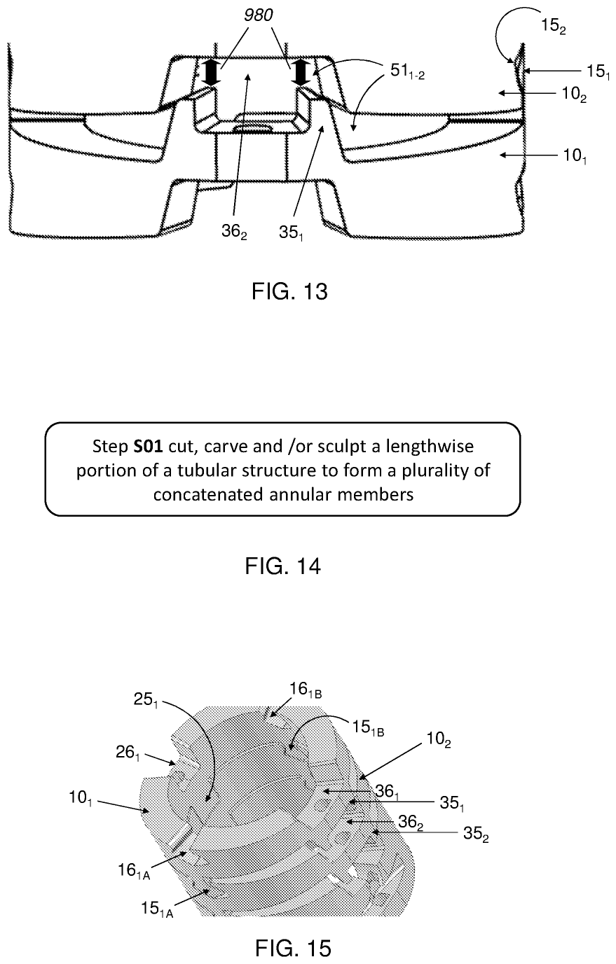

FIG. 13 illustrates, in greater detail, the structure and function of the first guiding arrangements between the annular members 10₁, 10₂, comprising guiding projection 35 and guiding recess 36 (specifically projection 35₁ of first annular member 10₁ and recess 36₂of second annular member 10₂). When the bending portion 99 is flexed as recess 16₂ pivots about projection 15₁, and annular members 10₁, 10₂ are made to pivot relative to each other, gap 51₁₋₂ is closed, as indicated by arrows 980. When the bending portion is unflexed, i.e., the bending portion returns towards being unflexed, the gap 51₁₋₂ is reopened. In embodiments, the flexing and pivoting are limited by a structural pivot limit. In the example of FIG. 13, the pivot limit (in the favored direction of flexing) is defined by the structure of the first guiding arrangement, and by the closing of gap 51₁₋₂ and the guiding projection 35 contacts the guiding recess 36. In embodiments, the annular members are shaped such that the first guiding arrangement provides the pivot limit of the bending portion 99, while the pivoting of the concave-angle portions 18 of the pivot-recesses 16 about the respective apex points 45 of the pivot-projections 15 do not provide the pivot limit of the bending portion 99. In other words, during pivoting, the guiding projection 35 contacts the guiding recess 36 before projection line segment B₁₅ (see FIG. 5) contacts the recess line segment B₁₆ and fully closes the gap 53 between B₁₅ and B₁₆. In embodiments, the annular members 10 are shaped such that projection line segment B₁₅ cannot contact the recess line segment B₁₆ because the pivoting is first stopped by the pivot-limiting design of the first guiding arrangement of FIG. 13.

A method is disclosed, according to embodiments, for producing a bendable section 99 for a tubular structure 40. As shown in the flowchart of FIG. 14, the method comprises:

Step S01 cut, carve and/or sculpt a lengthwise portion of a tubular structure 40 to form a plurality of concatenated annular members 10. According to the method, each annular member 10 is shaped by the at least one of cutting, carving and sculpting to comprise one or more axial pivot-projections 15 and one or more corresponding pivot-recesses 16 axially aligned therewith. Each of the one or more pivot-projections 15 is shaped to include an apex point 45 defining a vertex of a convex-angle portion 19 of the pivot-projection 15, each one of the one or more corresponding pivot-recesses 16 being shaped to surround a volume having an apex point 46 defining a vertex of a concave-angle portion 18 of the pivot-recess 16. For each pair of consecutive annular members 10₁, 10₂shaped by the at least one of cutting, carving and sculpting, the one or more pivot-projections 15₁ of a first annular member 10₁ are pivotably engaged with the one or more corresponding pivot-recesses 16₂ of a second annular member 10₂.

According to some embodiments, a pivoting of the first and second annular members 10₁, 10₂ relative to each other so as to bend the bendable section 99 includes a pivoting of the respective concave-angle portions 18 of the one or more corresponding pivot-recesses 16₂ of the second annular member 10₂ about the respective apex points 45 of the one or more pivot-projections 15₁ of the first annular member 10₁.

In some embodiments, the one or more axial pivot-projections 15 include exactly one pivot-projection 15, the one or more corresponding recesses 16 include exactly one corresponding pivot-recess 16, and the exactly one pivot-projection 15₁ of the first annular member 10₁ is pivotably engaged with the exactly one corresponding pivot-recess 16₂ of the second annular member 10₂. In some embodiments, as shown, e.g., in FIGS. 2D and 15, the one or more axial pivot-projections 15 include exactly two pivot-projections 15 that are diametrically opposed from each other, the one or more corresponding recesses 16 include exactly two corresponding pivot-recesses 16 that are diametrically opposed from each other, and the exactly two pivot-projections 15_{1A}, 15_{1B} of the first annular member 10₁ are respectively pivotably engaged with the exactly two corresponding pivot-recesses 16_{2A}, 16_{2B} of the second annular member 10₂.

In some embodiments, the convex angles α of the convex-angle portions 19 of the pivot-projections 15 are smaller than respective conjugate angles β of the concave angles β′ of the concave-angle portions 18 of the corresponding pivot-recesses 16.

In some embodiments, the at least one of cutting, carving and sculpting includes laser-cutting.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. An articulated arm comprising a tubular structure that includes:
a bendable section comprising a plurality of concatenated annular members,
each annular member comprising a first pair of axial projections and a corresponding first pair of recesses axially aligned therewith, each projection being shaped to include an apex point defining a vertex of a convex-angle portion of the projection, each corresponding recess being shaped to surround a volume having an apex point defining a vertex of a concave-angle portion of the recess,
the plurality of annular members being arranged in the bendable section such that for each pair of consecutive annular members, the projections of a first annular member of said pair are pivotably engaged with corresponding recesses of a second annular member of said pair,
wherein a pivoting of the first and second annular members relative to each other so as to facilitate bending of the bendable section includes a pivoting of the respective concave-angle portions of the corresponding recesses of the second annular member about the respective apex points of the projections of the first annular member;
wherein each annular member comprises a second pair of axial projections and a corresponding second pair of recesses axially aligned therewith, wherein, for each pair of consecutive annular members, the second-pair projections of the first annular member are slidably engaged with corresponding second-pair recesses of the second annular member to form respective guiding arrangements, a first guiding arrangement being effective to define a pivot limit of the first and second annular members in a direction of the flexing.

2. (canceled)

3. The articulated arm of claim 1, wherein when the respective concave-angle portions pivot about the respective apex points of the projections, the apex points of the projections are in direct contact with the respective vertices of the concave-angle portions.

4. (canceled)

5. (canceled)

6. The articulated arm of claim 1, wherein the projections and/or recesses are respectively devoid of axes of symmetry.

7. (canceled)

8. The articulated arm of claim 1, wherein the pivoting of the first and second annular members relative to each other is substantially without backlash and/or friction in the recesses.

9. (canceled)

10. (canceled)

11. The articulated arm of claim 1, wherein the pivoting of the first and second annular members relative to each other is not limited by a pivot limit of the pivoting of the concave-angle portions of the corresponding recesses of the second annular member about the respective apex points of the projections of the first annular member.

12. (canceled)

13. (canceled)

14. The articulated arm of claim 1, wherein the articulated arm comprises a first bendable section comprising a respective plurality of concatenated annular members having a first diameter, and a second bendable section comprising a respective plurality of concatenated annular members having a second diameter that is different than the first diameter.

15. The articulated arm of claim 1, wherein the pivoting of the first and second annular members relative to each other includes a relative pivoting between 5°-12°.

16. (canceled)

17. The articulated arm of claim 14, wherein bending either one of the first and second bendable sections includes bending the respective bendable section by at least 150°, or at least 180°.

18. (canceled)

19. An articulated arm comprising a tubular structure that includes:
a bendable section comprising a plurality of concatenated annular members,
each annular member comprising one or more axial pivot-projections and one or more corresponding pivot-recesses axially aligned therewith, each of the one or more pivot-projections being shaped to include an apex point defining a vertex of a convex-angle portion of the pivot-projection, each one of the one or more corresponding pivot-recesses being shaped to surround a volume having an apex point defining a vertex of a concave-angle portion of the pivot-recess,
the plurality of annular members being arranged in the bendable section such that for each pair of consecutive annular members, the one or more pivot-projections of a first annular member are pivotably engaged with the one or more corresponding pivot-recesses of a second annular member,
wherein a pivoting of the first and second annular members relative to each other so as to bend the bendable section includes a pivoting of the respective concave-angle portions of the one or more corresponding pivot-recesses of the second annular member about the respective apex points of the one or more pivot-projections of the first annular member;
wherein each annular member comprises one or more axial guide-projections and one of more corresponding guide-recesses axially aligned therewith, wherein the one or more guide-projections of the first annular member are slidably engaged with corresponding one or more guide-recesses of the second annular member to form one or more respective guiding arrangements, a first guiding arrangement being effective to define a pivot limit of the first and second annular members in a direction of the flexing.

20. The articulated arm of claim 19, wherein the one or more axial pivot-projections include exactly one pivot-projection, the one or more corresponding recesses include exactly one corresponding pivot-recess, and the exactly one pivot-projection of the first annular member is pivotably engaged with the exactly one corresponding pivot-recess of the second annular member.

21. The articulated arm of claim 19, wherein the one or more axial pivot-projections include exactly one pivot-projection, the one or more corresponding pivot-recesses include exactly two pivot-recesses diametrically opposed from each other, and the exactly one pivot-projection of the first annular member is pivotably engaged with one of the two pivot-recesses of the second annular member.

22. (canceled)

23. The articulated arm of claim 19, wherein when the respective concave-angle portions pivot about the respective apex points of the pivot-projections, the apex points of the pivot-projections are in direct contact with the respective vertices of the concave-angle portions.

24. (canceled)

25. (canceled)

26. The articulated arm of claim 19, wherein the pivot-projections and or pivot-recesses are respectively devoid of axes of symmetry.

27. (canceled)

28. The articulated arm of claim 19, wherein the pivoting of the first and second annular members relative to each other is substantially without backlash and/or friction in the pivot-recess.

29. (canceled)

30. (canceled)

31. The articulated arm of claim 19, wherein the pivoting of the first and second annular members relative to each other is not limited by a pivot limit of the pivoting of the concave-angle portions of the corresponding one or more pivot-recesses of the second annular member about the respective apex points of the one or more pivot-projections of the first annular member.

32. (canceled)

33. The articulated arm of claim 19, wherein the articulated arm comprises a first bendable section comprising a respective plurality of concatenated annular members having a first diameter, and a second bendable section comprising a respective plurality of concatenated annular members having a second diameter that is different than the first diameter.

34. The articulated arm of claim 19, wherein the pivoting of the first and second annular members relative to each other includes a maximum relative pivoting between 5°-12°.

35. (canceled)

36. The articulated arm of claim 33, wherein bending either one of the first and second bendable sections includes bending the respective bendable section by at least 150°, or at least 180°.

37. (canceled)

38. An annular constituent element for a bendable section of a tubular structure, the annular element comprising one or more axial projections and one or more corresponding recesses axially aligned therewith, each of the one or more projections being shaped to include an apex point defining a vertex of a convex-angle portion of the projection, each one of the one or more corresponding recesses being shaped to surround a volume having an apex point defining a vertex of a concave-angle portion of the recess;
wherein the annular constituent element comprises a second pair of axial projections and a corresponding second pair of recesses axially aligned therewith, configured for being slidably engaged with corresponding second-pair recesses of a second annular constituent element to form respective guiding arrangements, a first guiding arrangement being effective to define a pivot limit of the first and second annular members in a direction of the flexing.

39. The annular constituent element of claim 38, wherein the one or more axial projections comprise respective corresponding inner and outer surfaces, and each respective outer surface has an area smaller than an area of a corresponding axially-aligned-therewith recess.

40. (canceled)

41. (canceled)

42. The annular constituent element of claim 38, wherein the one or more axial projections and/or recesses are respectively devoid of axes of symmetry.

43-49. (canceled)
